(19) United States
(12) Patent Application Publication
(10) Pub. No.: US 2003/0114807 A1
(43) Pub. Date: Jun. 19, 2003

(54) Disposable absorbent article

(75) Inventors: Richard L. Underhill, Neenah, WI (US); Michael J. Niemeyer, Appleton, WI (US); Marianne K. Leick, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/017,166

(22) Filed: Dec. 14, 2001

(51) Int. Cl. A61F 13/15

(52) U.S. Cl. 604/361; 604/368

(57) ABSTRACT

A disposable absorbent article having a generally liquid permeable liner, an outer cover and an absorbent body between the liner and outer cover for absorbing liquid body waste penetrating the liner. In one embodiment, the absorbent body has a first zone and a second zone, with the first zone having a lower absorbent capacity per unit weight than the second zone and being capable of facilitating the flow of liquid body waste from the first zone back through the liner. In another embodiment, the absorbent body comprises a mixture of hydrophilic fibers and superabsorbent material. The mixture has a concentration of superabsorbent material of between about one percent and about fifteen percent by weight of the mixture to facilitate the flow of liquid body waste from the absorbent body back through the liner.

### BACKGROUND OF THE INVENTION

The present invention relates to disposable absorbent articles, such as those used as personal care products, and more particularly to such a disposable absorbent article which provides an indication of wetness to the wearer upon the release of liquid body waste into the article.

Disposable absorbent articles find widespread use as personal care products such as diapers, children's toilet training pants and other infant and child care products, adult incontinence garments and other adult care products, sanitary napkins and other feminine care products and the like, as well as surgical bandages and sponges. These articles absorb and contain body waste and are intended to be discarded after a limited period of use; i.e., the articles are not intended to be laundered or otherwise restored for reuse. Conventional disposable absorbent articles comprise an absorbent body disposed between an inner layer adapted for contacting the wearer's skin and an outer layer for inhibiting liquid waste absorbed by the absorbent body from leaking out of the article. The inner layer of the absorbent article is typically liquid permeable to permit body waste to pass therethrough for absorption by the absorbent body.

For example, children's toilet training pants serve as a training aid as a child transitions from diapers to underpants. Conventional toilet training pants are three-dimensional articles, similar to underpants in appearance, but constructed with a liquid permeable inner layer and an absorbent body to provide the absorbent function of disposable absorbent articles. An initial step in the toilet training process is for the child to recognize when urination occurs. However, where the training pants quickly and effectively draw urine away from the skin and retain the urine in the absorbent body, the inner layer of the pants remains dry and comfortable against the child's skin. As a result, there is little or no recognizable indication to the child that he or she has urinated. To this end, it is desirable to provide some indication to the child that the child has urinated in the training pants.

Therefore, despite advancements in the construction of disposable absorbent articles, there continues to be a need for relatively easily constructed disposable absorbent articles capable of indicating to the wearer that a release of liquid body waste has occurred.

### SUMMARY OF THE INVENTION

In general, a disposable absorbent article of the present invention comprises a generally liquid permeable liner adapted for contiguity with the wearer's skin, an outer cover, and an absorbent body between the liner and the outer cover for absorbing liquid body waste penetrating the liner. The absorbent body comprises a first zone and a second zone. The first zone has a lower absorbent capacity per unit weight than the second zone and facilitates the flow of liquid body waste from the first zone back through the liner for indicating to the wearer the release of liquid body waste into the article.

In another embodiment, a disposable absorbent article generally comprises a generally liquid permeable liner adapted for contiguity with the wearer's skin, an outer cover and an absorbent body between the liner and the outer cover for absorbing liquid body waste. The absorbent body comprises a mixture of hydrophilic fibers and superabsorbent material. The mixture has a concentration of superabsorbent material of between about one percent and about seven percent by weight of the mixture to facilitate the flow of liquid body waste from the absorbent body back through the liner for indicating to the wearer the release of liquid body waste into the article.

In yet another embodiment, the disposable absorbent article generally comprises a generally liquid permeable liner adapted for contiguity with the wearer's skin, an outer cover and an absorbent body between the liner and outer cover for absorbing liquid body waste penetrating the liner. The absorbent body has a width of at least about sixty percent of the width of the article and comprises a mixture of hydrophilic fibers and superabsorbent material. The mixture has a concentration of superabsorbent material therein of between about one percent and about fifteen percent by weight of the mixture to facilitate the flow of liquid body waste from the absorbent body back through the liner for indicating to the wearer the release of liquid body waste into said article.

Toilet training pants of the present invention generally comprise an anterior region, a posterior region and a crotch region disposed longitudinally therebetween. The anterior region, posterior region and crotch region are integrally formed and configured to define a central waist opening and a pair of leg openings of said pants, with the crotch region extending generally laterally between said leg openings. A generally liquid permeable liner extends from the anterior region through the crotch region to the posterior region of the pants and is adapted for contiguity with the wearer's skin. An absorbent body lies between the liner and an outer cover for absorbing liquid body waste penetrating the liner. The absorbent body comprises a first zone and a second zone. The first zone has a lower absorbent capacity per unit weight than the second zone and is capable of facilitating the flow of liquid body waste from the first zone back through the liner for indicating to the wearer the release of liquid body waste into said training pants.

In general, one method of the present invention for manufacturing an absorbent body for a disposable absorbent article of the type having a liner, an outer cover and an absorbent body between the liner and outer cover for absorbing liquid body waste penetrating the liner comprises concurrently depositing hydrophilic fibers and superabsorbent material on a surface for accumulation thereon to form the absorbent body. The hydrophilic fibers are deposited generally uniformly across the absorbent body. The concentration of superabsorbent material deposited in a wetness indicating zone of the absorbent body is lower than the concentration of superabsorbent material deposited in absorbent zones of the absorbent body.

Another method of manufacturing an absorbent body for a disposable absorbent article of the type having a liner, an outer cover and an absorbent body between the liner and outer cover for absorbing liquid body waste penetrating the liner generally comprises concurrently depositing hydrophilic fibers and superabsorbent material on a surface for accumulation thereon to form the absorbent body. The concentration of hydrophilic fibers deposited in a wetness indicating zone of the absorbent body is greater than the concentration of fibers deposited in absorbent zones of the absorbent body. The concentration of superabsorbent material deposited in the wetness indicating zone is lower than the concentration of superabsorbent material deposited in each absorbent zone.

Other aspects and features of this invention will be in part apparent and in part pointed out hereinafter.

### BRIEF DESCRIPTION OF THE DRAWINGS

FIG. 1 is a side perspective of children's toilet training pants of the present invention;

FIG. 2 is a top plan of the training pants of FIG. 1 with the pants shown unfastened and laid flat and portions of the pants broken away to reveal internal construction thereof;

FIG. 3 is a schematic, separated cross-section of the training pants of FIG. 1 taken transversely through a crotch region of the pants;

FIG. 4 is a graph of flow back versus time after liquid delivery resulting from an experiment conducted on sample absorbent bodies of the type shown in FIG. 3;

FIG. 5 is a schematic, separated cross-section taken transversely through a crotch region of children's toilet training pants of a second embodiment of the present invention;

FIG. 6 is a graph of flow back versus time after liquid delivery resulting from an experiment conducted on sample absorbent bodies of the type shown in FIG. 5;

FIG. 7 is a schematic, separated cross-section taken transversely through a crotch region of children's toilet training pants of a third embodiment of the present invention; and

FIG. 8 is a graph of flow back versus time after liquid delivery resulting from an experiment conducted on sample absorbent bodies of the type shown in FIG. 7.

Corresponding reference characters indicate corresponding parts throughout the drawings.

### DEFINITIONS

Within the context of this specification, each term or phrase below will include the following meaning or meanings:

(a) “Bonded” refers to the joining, adhering, connecting, attaching, or the like, of two elements. Two elements will be considered to be bonded together when they are bonded directly to one another or indirectly to one another, such as when each is directly bonded to intermediate elements.

(b) “Film” refers to a thermoplastic film made using a film extrusion and/or foaming process, such as a cast film or blown film extrusion process. The term includes apertured films, slit films, and other porous films which constitute liquid transfer films, as well as films which do not transfer liquid.

(c) “Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

(d) “Layer” when used in the singular can have the dual meaning of a single element or a plurality of elements.

(e) “Liquid impermeable,” when used in describing a layer or multi-layer laminate means that liquid body waste, such as urine, will not pass through the layer or laminate, under ordinary use conditions, in a direction generally perpendicular to the plane of the layer or laminate at the point of liquid contact.

(f) “Liquid permeable” refers to any material that is not liquid impermeable.

(g) “Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

(h) “Non-woven” and “non-woven web” refer to materials and webs of material which are formed without the aid of a textile weaving or knitting process.

(i) “Pliable” refers to materials which are compliant and which will readily conform to the general shape and contours of the wearer's body.

(j) “Spunbond” refers to small diameter fibers which are formed by extruding molten thermoplastic material as filaments from a plurality of fine capillaries of a spinnerette having a circular or other configuration, with the diameter of the extruded filaments then being rapidly reduced by a conventional process such as that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, more particularly, between about 0.6 and about 10.

(k) “Superabsorbent” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers.

(l) “Surge Layer” refers to a layer capable of rapidly accepting and temporarily holding liquid body waste to decelerate and diffuse a surge or gush of liquid body waste and to subsequently slowly release the liquid body waste therefrom into another layer or layers.

(m) “Thermoplastic” describes a material which softens when exposed to heat and which substantially returns to a non-softened condition when cooled to room temperature.

(n) “Three dimensional” refers to a garment similar to underwear, shorts or pants in that it has continuous leg and waist openings that are bounded by material of which the garment is made. The garment may or may not have manually tearable seams.

### DETAILED DESCRIPTION OF THE PREFERRED EMBODIMENTS

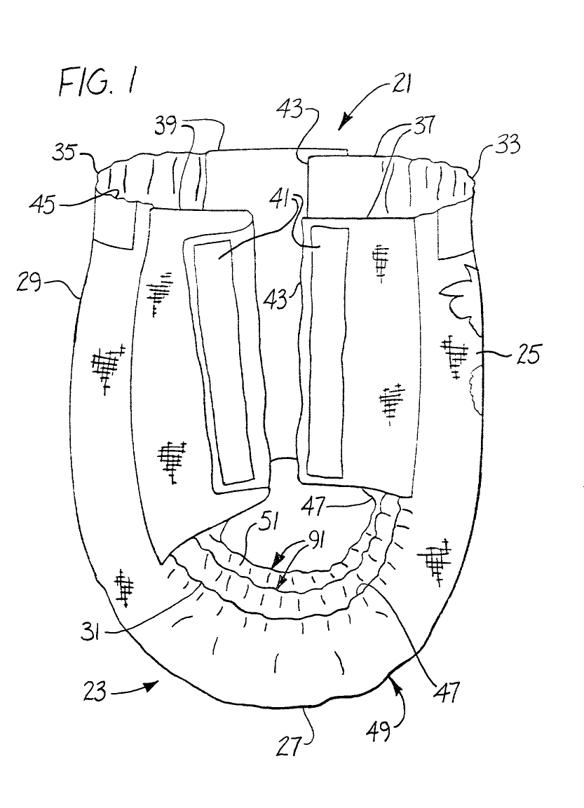

Referring now to the drawings and in particular to FIG. 1, a disposable absorbent article of the present invention is shown in the form of children's toilet training pants and is indicated in its entirety by the reference numeral 21. As used herein, a disposable absorbent article refers to an article which may be placed against or in proximity to the body (i.e., contiguous to the body) of the wearer to absorb and contain various liquid waste discharged from the body. Such articles are intended to be discarded after a limited period of use instead of being laundered or otherwise restored for reuse. It is understood that the present invention is applicable to various other disposable absorbent articles, such as diapers and other infant and child care products, adult incontinence garments and other adult care products, sanitary napkins and other feminine care products and the like, as well as surgical bandages and sponges, without departing from the scope of the present invention.

By way of illustration only, various materials and methods for constructing training pants 21 are disclosed in PCT Patent Application WO 00/37009 published Jun. 29, 2000 by A. Fletcher et al; U.S. Pat. No. 4,940,464 issued Jul. 10, 1990 to Van Gompel et al.; and U.S. Pat. No. 5,766,389 issued Jun. 16, 1998 to Brandon et al., which are incorporated herein by reference.

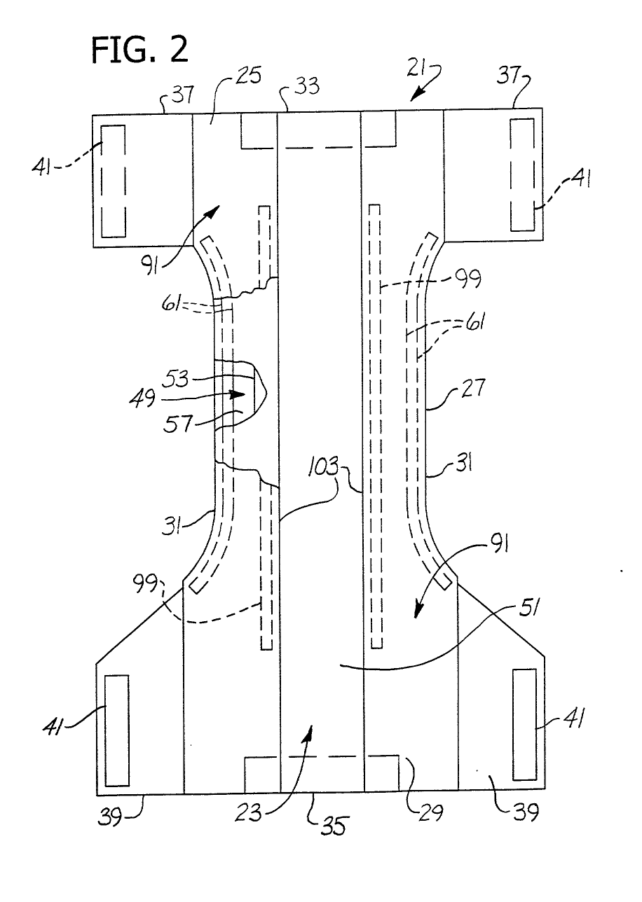

The training pants 21 of the illustrated embodiment generally comprise a central absorbent assembly 23 extending longitudinally from an anterior region 25 of the training pants through a crotch region 27 to a posterior region 29 of the training pants. As illustrated in FIG. 2, the central absorbent assembly 23 is generally rectangular and has laterally opposite side edges 31 and longitudinally opposite front and rear waist edges, respectively designated 33 and 35. Front and rear side panels 37, 39, respectively, are secured to the central absorbent assembly 23 as will be described later herein and extend laterally outward therefrom respectively at the anterior and posterior regions 25, 29 of the training pants 21.

To form the three-dimensional training pants 21, corresponding front and rear side panels 37, 39 (e.g., the front left side panel and the rear left side panel) are refastenably secured together, using fastening assemblies 41, along generally vertical seams 43. Alternatively, the front and rear side panels 37, 39 may be permanently secured together, such as by ultrasonic bonding, or they may be formed integrally with each other and with the central absorbent assembly 23. Securing the side panels 37, 39 together provides a central waist opening 45 and a pair of laterally spaced leg openings 47 of the training pants 21. The training pants 21 are worn by inserting the wearer's feet through the waist opening 45 and the respective leg openings 47; grasping the training pants near the waist opening; and then pulling the pants up along the wearer's legs until the crotch region 27 of the training pants fits snugly against the crotch of the wearer.

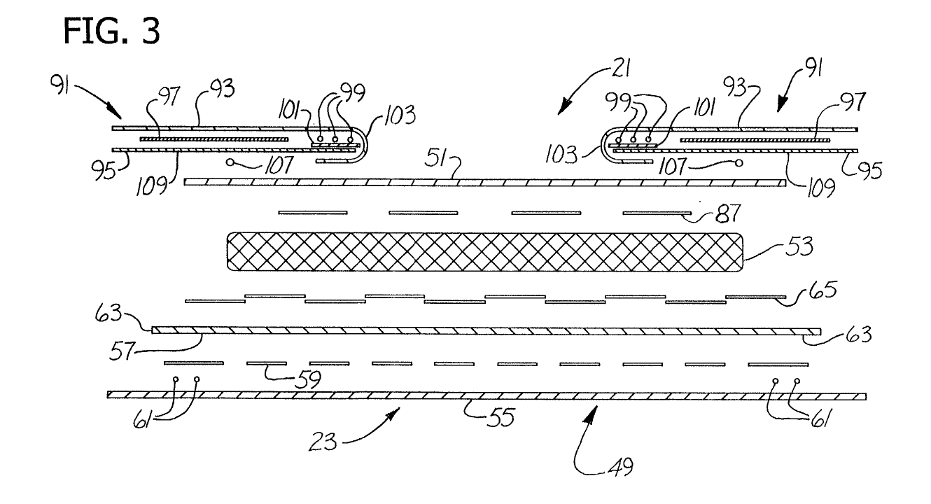

With reference to FIG. 3, the central absorbent assembly 23 of the training pants 21 comprises an outer cover, generally indicated at 49, a bodyside liner 51 and an absorbent body 53 disposed between the outer cover and the liner. The outer cover 49 can be elastic, stretchable or non-stretchable and is desirably a multi-layered laminate structure of which at least one of the layers is liquid impermeable. For example, the outer cover 49 of the illustrated embodiment is of two-layer construction, including an outer layer 55 constructed of a liquid permeable material and an inner layer 57 constructed of a liquid impermeable material joined together by a laminate adhesive 59. Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. It is understood that the outer cover 49 may instead be constructed of a single layer of impermeable material without departing from the scope of this invention.

The liquid permeable outer layer 55 of the outer cover 49 can be any suitable material and is desirably one that provides a generally cloth-like texture. One example of such a material is a 20 gsm (grams per square meter) spunbond polypropylene non-woven web. The outer layer 55 may also be constructed of the same materials from which the bodyside liner 51 is constructed as described later herein. Also, while it is not a necessity for the outer layer 55 of the outer cover 49 to be liquid permeable, it is desired that it provide a relatively cloth-like texture to the wearer.

The liquid impermeable inner layer 57 of the outer cover 49 can be either vapor permeable (i.e., “breathable”) or vapor impermeable. The inner layer 57 is desirably manufactured from a thin plastic film, although other flexible liquid impermeable materials may also be used. The liquid impermeable inner layer 57 (or the liquid impermeable outer cover 49 where the outer cover is of a single-layer construction) inhibits liquid body waste from leaking out of the pants and wetting articles, such as bed sheets and clothing, as well as the wearer and care giver. A suitable liquid impermeable material for such use is a 0.02 millimeter polyethylene film commercially available from Huntsman Packaging of Newport News, Va., U.S.A.

Where the outer cover 49 is of single-layer construction, it can be embossed and/or matte finished to provide a more cloth-like appearance. As earlier mentioned, the liquid impermeable layer of the outer cover 49 can permit vapors to escape from the pants 21 while preventing liquids from passing therethrough. A suitable liquid impermeable, vapor permeable material is composed of a microporous polymer film or a non-woven fabric which has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn., U.S.A.

Leg elastic members 61 are secured between the outer and inner layers 55, 57 of the outer cover 49, such as by being bonded therebetween by the laminate adhesive 59, generally adjacent laterally outer edges 63 of the inner layer of the outer cover. Alternatively, the leg elastic members 61 may be disposed between the outer cover 49 and the bodyside liner 51, or between other layers of the pants 21. A wide variety of elastic materials may be used for the leg elastic members 61. As is well known to those skilled in the art, suitable elastic materials include sheets, strands or ribbons of natural rubber, synthetic rubber, or thermoplastic elastomeric polymers. The elastic materials can be stretched and secured to a substrate, secured to a gathered substrate, or secured to a substrate and then elasticized or shrunk, for example with the application of heat, such that elastic retractive forces are imparted to the substrate. For example, one suitable elastic material is a dry-spun coalesced multifilament spandex elastomeric thread sold under the trade name LYCRA® and available from E. I. duPont de Nemours and Company, Wilmington, Del., U.S.A.

The absorbent body 53 is somewhat rectangular and is desirably -t -constructed to be generally compressible, pliable, non-irritating to the wearer's skin and capable of absorbing and retaining liquid body waste, such as urine. The absorbent body 53 is desirably comprises a mixture of hydrophilic fibers, such as cellulosic fluff, and a high-absorbency material commonly known as superabsorbent material. More particularly, the absorbent body 53 desirably comprises a mixture of cellulosic fluff, such as wood pulp fluff, and superabsorbent hydrogel-forming particles. One suitable type of wood pulp fluff is identified with the trade designation CR1654, available from U.S. Alliance, Childersburg, Ala., U.S.A., and is a bleached, highly absorbent sulfate wood pulp containing primarily soft wood fibers. However, the wood pulp fluff can be exchanged with other hydrophilic fiber materials, such as synthetic, polymeric, or meltblown fibers or with a combination of meltblown fibers and natural fibers.

Suitable superabsorbent materials can be selected from natural, synthetic, and modified natural polymers and materials. The superabsorbent materials can be inorganic materials, such as silica gels, or organic compounds, such as cross-linked polymers. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany.

The relative concentrations of wood pulp fluff and superabsorbent material from which the absorbent body 53 is constructed desirably renders the absorbent body capable of maintaining about 1 gram of urine therein per 1 gram of the mixture for a prolonged duration after the wearer has urinated in the training pants 21, and more desirably allows the absorbent body to retain about 1.5 grams of urine therein per 1 gram of mixture. The urine maintained therein may be absorbed by the superabsorbent material or the wood pulp fluff, or it may be unabsorbed and entrained in the interstitial spacings between hydrophilic fibers such that it is capable of flowing back out of the absorbent body.

As an example, the duration during which the desired amount of urine remains capable of flowing back out of the absorbent body 53 is at least about one minute, is more desirably about five minutes, and is even more desirably about ten minutes. The concentration of superabsorbent material present in the absorbent body 53 desirably comprises about one percent to about fifteen percent by weight of the absorbent body, and more desirably comprises about one percent to about seven percent by weight of the absorbent body. As a comparison, the concentration of superabsorbent material present in an absorbent body (not shown) of conventional toilet training pants comprises in the range of about twenty percent to about fifty percent by weight, and is more typically about forty percent by weight of the absorbent body. The absorbent body 53 desirably has a density of about 0.10 to about 0.35 grams per cubic centimeter.

The absorbent body 53 may or may not be wrapped or encompassed by a suitable wrapping (not shown) which maintains the integrity and/or shape of the absorbent body. The absorbent body 53 overlays the inner layer 57 of the outer cover 49, extending laterally between the leg elastic members 61, and is secured to the inner layer, such as by being bonded thereto with adhesive 65. Although, it is contemplated that the absorbent body 53 may free from securement to the inner layer 57 of the outer cover 49 without departing from the scope of this invention. The width of the absorbent body is desirably at least about sixty percent of the width of the training pants 21. For example, the narrowest width of the training pants 21, such as in the crotch region 27 thereof between the leg openings 47, is about 5.5 inches and the width of the absorbent body 53 is about 4.5 inches. Hence the width of the absorbent body 53 of the illustrated embodiment is about 82% of the narrowest width of the training pants 21. It is understood, however, that the widths of the absorbent body 53 and the training pants 21 may vary.

Still referring to FIG. 3, the bodyside liner 51 overlays the absorbent body 53 to isolate the wearer's skin from liquid body waste retained by the absorbent body and is secured to at least a portion of the absorbent body, such as by being bonded thereto with adhesive 87. The liner 51 further extends beyond the absorbent body 53 to overlay a portion of the inner layer 57 of the outer cover 49, particularly in the crotch region 27 of the pants 21, and is secured thereto, such as by being bonded thereto by adhesive 65, to substantially enclose the absorbent body between the outer cover and the liner about the periphery of the absorbent body. Although the bodyside liner 51 shown in FIG. 3 is slightly narrower than the outer cover 49, it is understood that the liner and outer cover may be of the same dimensions, or the liner may be sized larger than the outer cover, without departing from the scope of this invention. It is also contemplated that the liner 51 may not extend beyond the absorbent body 53 and may not be secured to the outer cover 49 and/or to the absorbent body 53. The bodyside liner 51 is desirably compliant, soft feeling, and non-irritating to the wearer's skin and can be less hydrophilic than the absorbent body 53 to provide a relatively dry surface to the wearer and permit liquid body waste to readily penetrate through its thickness.

The bodyside liner 51 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and non-woven fabrics can be used for the bodyside liner 51. For example, the liner 51 can be composed of a meltblown or spunbonded web of polyolefin fibers. Alternatively, the liner 51 can be a bonded-carded web composed of natural and/or synthetic fibers. The bodyside liner 51 can also be composed of a substantially hydrophobic material, and the hydrophobic material can, optionally, be treated with a surfactant or otherwise processed to impart a desired level of wetability and hydrophilicity. For example, the material can be surface treated with about 0.45 weight percent of a surfactant mixture including AHCOVEL® N-62 available from Uniqema, Inc., a division of ICI of New Castle, Del., U.S.A, and GLUCOPON® 220UP available from Cognis Corporation of Ambler, Pa., U.S.A, in an active ratio of 3:1. The surfactant can be applied by any conventional means, such as spraying, printing, brush coating or the like. The surfactant can be applied to the entire liner 51 or it can be selectively applied to particular sections of the liner.

A particularly suitable bodyside liner 51 is constructed of a non-woven bicomponent web having a basis weight of about 27 gsm. The non-woven bicomponent can be a spunbonded bicomponent web, or a bonded-carded bicomponent web. Suitable bicomponent staple fibers include a polyethylene/polypropylene bicomponent fiber available from CHISS® Corporation, Osaka, Japan. In this particular bicomponent fiber, the polypropylene forms the core and the polyethylene forms the sheath of the fiber. Fibers having other orientations, such as multi-lobe, side-by-side, end-to-end may be used without departing from the scope of the invention. Also, although the outer cover 49 and bodyside liner 51 of the central absorbent assembly 23 can include elastomeric materials, it is contemplated that the central absorbent assembly may instead be generally inelastic, wherein the outer cover, the bodyside liner and the absorbent body 53 are composed of materials which are generally non-elastomeric.

It is also contemplated that other layers (not shown) may lie between the liner 51 and the absorbent 53. For example, a surge layer similar to that disclosed in U.S. Pat. Nos. 5,490,846 and 5,562,650, the entire disclosures of which are incorporated herein by reference, may lie therebetween for quickly accepting surges of liquid body waste which penetrate the liner, temporarily holding the liquid body waste therein and then slowly releasing the liquid body waste to the absorbent body, without departing from the scope of this invention.

The front and rear side panels 37, 39 of the training pants 21 are bonded to the central absorbent assembly 23 at the respective anterior and posterior regions 25, 29 of the pants and extend outward beyond the laterally opposite edges 31 of the assembly. For example, the front side panels 37 of the illustrated embodiment are secured to the inner layer 57 of the outer cover 49, such as by being bonded thereto by adhesive (not shown), by thermal bonding or by ultrasonic bonding. These side panels 37 may also be secured to the outer layer 55 of the outer cover 49, such as by being bonded thereto by adhesive (not shown), by thermal bonding or by ultrasonic bonding. The rear side panels 39 are secured to the outer and inner layers 55, 57 of the outer cover 49, at the posterior region 29 of the training pants 21, in substantially the same manner as the front side panels 37. Alternatively, the side panels 37, 39 may be formed integrally with the central absorbent assembly 23, such as by being formed integrally with the outer cover 49, the bodyside liner 51 or other layers of the pants 21.

For improved fit and appearance, the side panels 37, 39 desirably have an average length measured parallel to the longitudinal axis of the training pants 21 that is about 20 percent or greater, and more desirably about 25 percent or greater, of the overall length of the training pants, also measured parallel to the longitudinal axis. For example, for training pants 21 having an overall length of about 54 centimeters, the side panels 37, 39 desirably have an average length of about 10 centimeters or greater, and more desirably an average length of about 15 centimeters. Each of the side panels 37, 39 can be constructed of one or more individual, distinct pieces of material. For example, each side panel 37, 39 can include first and second side panel portions (not shown) joined at a seam (not shown), with at least one of the portions including an elastomeric material. Alternatively, each individual side panel 37, 39 can be constructed of a single piece of material folded over upon itself along an intermediate fold line (not shown).

The side panels 37, 39 desirably include an elastic material capable of stretching laterally. Suitable elastic materials, as well as one described process for incorporating elastic side panels 37, 39 into training pants 21, are described in the following U.S. Pat. Nos. 4,940,464 issued Jul. 10, 1990 to Van Gompel et al.; 5,224,405 issued Jul. 6,1993 to Pohjola; 5,104,116 issued Apr. 14, 1992 to Pohjola; and 5,046,272 issued Sep. 10,1991 to Vogt et al.; all of which are incorporated herein by reference. As an example, suitable elastic materials include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and European Patent Application No. EP 0 217 032 published on Apr. 8,1987 in the names of Taylor et al.; all of which are incorporated herein by reference. Alternatively, the side panels 37, 39 may include other woven or non-woven materials, such as those described above as being suitable for the outer cover 49 or bodyside liner 51, or stretchable but inelastic materials.

Containment flaps, generally indicated at 91 (FIG. 3), are secured to the body side liner 51 in generally parallel, spaced relation with each other laterally inward of the leg openings 47 to provide a barrier against the flow of urine to the leg openings. The containment flaps 91 extend longitudinally from the anterior region 25 of the training pants 21, through the crotch region 27 to the posterior region 29 of the pants. Each containment flap 91 comprises a non-woven layer 93 and a film layer 95 secured to the non-woven layer, such as by being bonded thereto by adhesive 97. Flap elastics 99 are secured by suitable adhesive 101 between the non-woven layer 93 and the film layer 95 generally at a distal end 103 of the flap 91, with the non-woven layer 93 being folded over the flap elastics 99 and the film layer 95 at the distal end 103. The flap 91 is secured to the bodyside liner 51 by a seam of adhesive 107 to define a proximal end 109 of the flap.

The flap elastics 99 of the illustrated embodiment comprise three individual strands of elastomeric material extending longitudinally along the distal end 103 of the flap 91 in generally parallel, spaced relation with each other. One suitable elastic strand is a LYCRA® T151 940 decitex elastic which can be obtained from E. I. duPont de Nemours Co. of Wilmington, Del. The elastic strands are secured between the non-woven layer 93 and the film layer 95 while in an elastically contractible condition such that contraction of the strands gathers and shortens the distal end 103 of the containment flap 91. As a result, the elastic strands bias the distal end 103 of each flap 91 toward a position spaced from the proximal end 109 of the flap so that the flap extends away from the liner 51 in a generally upright orientation of the flap, especially in the crotch region 27 of the training pants 21, when the pants are fitted on the wearer. It is understood, however, that the containment flaps 91 may be omitted from the training pants 21 without departing from the scope of the invention.

In use, when the wearer of the training pants 21 urinates therein, urine penetrates the liner 51 and is received by the absorbent body 53. Because the concentration of superabsorbent material present in the absorbent body 53 is relatively low, a lower concentration of urine is absorbed by superabsorbent material, leaving a higher concentration of unabsorbed urine entrained in the interstitial spacing of the wood pulp fluff fibers for a prolonged duration. The prolonged maintenance of unabsorbed urine in the absorbent body 53 facilitates the flow of urine from the absorbent body back through the liner 51 to provide a prolonged feeling of wetness against the wearer's skin for indicating to the wearer that he or she has urinated.

### Experiment 1

An experiment was conducted to determine the amount of liquid capable of flowing back out of various absorbent body samples after one minute, after five minutes, and after ten minutes following liquid delivery. The experiment entailed the use of samples having a length of 6.75 inches, a width of five inches and a dry weight of about 16 grams. The samples were each composed of a homogeneous mixture of wood pulp fluff and superabsorbent material, with the concentration of superabsorbent material present in each sample respectively comprising five (Sample 1), ten (Sample 2), fifteen (Sample 3), twenty (Sample 4) or forty (Sample 5) percent by weight of the sample. The sample having a superabsorbent material concentration of about forty percent (i.e., Sample 5) simulated an absorbent body of conventional training pants. The dry weight of each sample was measured and only samples that weighed within three percent of the targeted sample weight were tested.

For each sample tested, the sample was placed between a pair of panels constructed of 10 mm thick plexiglass, with the top panel having a two inch diameter hole positioned centrally over the sample. A cylindrical tube having a two inch inner diameter and a height of about 85 mm was placed on the top panel over the opening. 100 ml±5 ml of 0.9% saline solution was poured into the tube and allowed to be taken into the sample. The sample was then retained between the panels for an additional time period corresponding to the desired duration after liquid delivery, e.g., one minute, five minutes or ten minutes. Thereafter, the sample was removed from between the panels.

Blotter paper, or papers where necessary, was placed against the sample and the sample was exposed to a vacuum pressure of about 0.5 pounds per square inch (e.g., about 3.45 kPa) for a duration of about two minutes to draw liquid from the sample into the blotter paper. The weight of the blotter paper was then measured and compared to the dry weight of the blotter paper to determine the amount of liquid drawn from the sample. The test was repeated five times for each sample type and the average amount of liquid drawn from the sample was determined from the five tests run for each sample type.

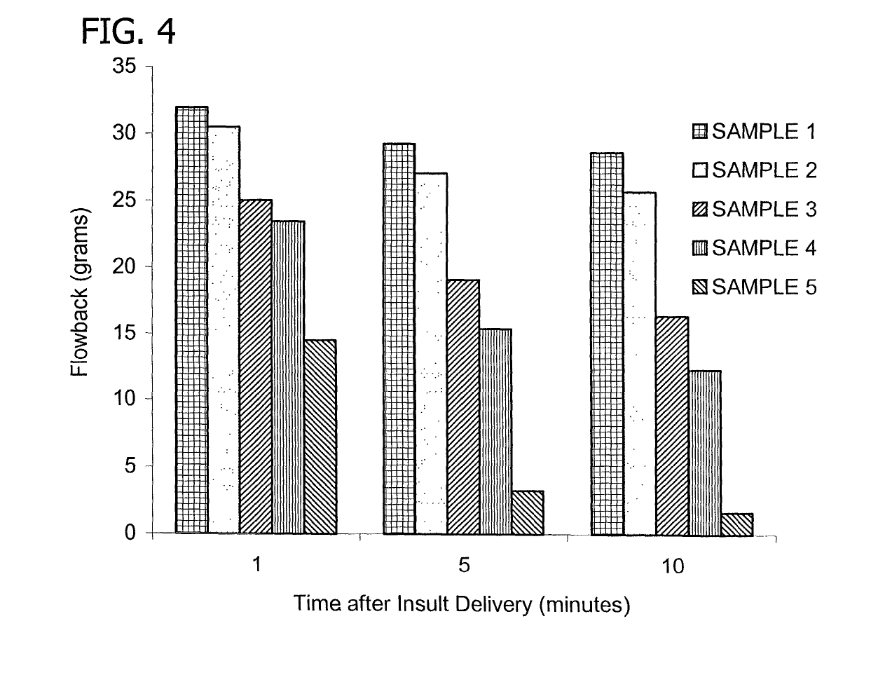

The results of the experiment are illustrated in FIG. 4. Absorbent body samples in which the concentration of superabsorbent material comprised five percent (i.e., Sample 1) or ten percent (i.e., Sample 2) by weight maintained (referred to as flow back in FIG. 4) more than about twenty-five grams of liquid for flow back out of the sample (i.e., more than about 1.5 grams per 1 gram of sample weight) for at least ten minutes; samples in which the concentration of superabsorbent material comprised fifteen percent by weight (i.e., Sample 3) maintained more than about fifteen grams of liquid for flow back out of the sample (e.g., more than about 1 gram per 1 gram of sample weight) for at least ten minutes; and samples in which the concentration of superabsorbent material comprised more than fifteen percent by weight (i.e., Samples 4 and 5) maintained less than fifteen grams of liquid for flow back out of the sample for at least ten minutes.

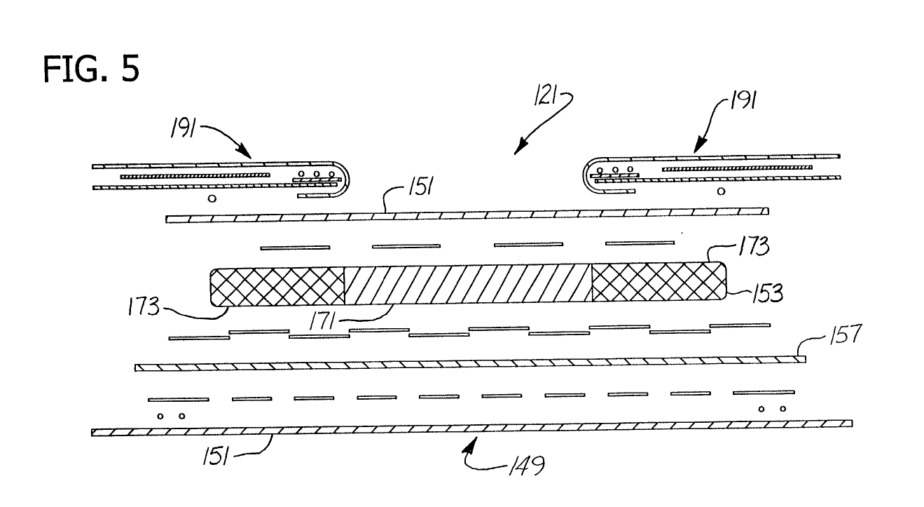

FIG. 5 illustrates training pants 121 of a second embodiment of the present invention similar to the training pants 21 of the first embodiment including a liner 151, an outer cover 149 having an outer layer 155 and an inner layer 157, an absorbent body 153 and containment flaps 191. The absorbent body 153 of this second embodiment comprises a central wetness indicating zone 171 (broadly, a first zone) and a pair of absorbent zones 173 (together broadly defining a second zone) disposed adjacent to the central wetness indicating zone on laterally opposite sides thereof. The central wetness indicating zone 171 desirably comprises about thirty percent to about seventy percent of the total width of the absorbent body 153, and more desirably about fifty percent of the total width of the absorbent body. As an example, the absorbent body 153 of the illustrated embodiment has a total width of about four inches, and the central wetness indicating zone 171 has a width of about two inches (thereby constituting about fifty percent of the absorbent body width).

It is contemplated that one absorbent zone 173 may fully surround the periphery of the central wetness indicating zone 171. It is also contemplated that the wetness indicating zone 171 may not be centrally positioned between the absorbent zones 173. The absorbent zones 173 may also be adjacent to but not in abutting relationship with the wetness indicating zone 171, such as by separated therefrom by an additional zone or layer of material. Also, the central wetness indicating zone 171 and the absorbent zones 173 may or may not be formed integrally and remain within the scope of this invention.

The absorbent body 153 is desirably constructed such that the central wetness indicating zone has a lower absorbent capacity per unit weight than the absorbent zones. The central wetness indicating zone is thus capable of maintaining a higher concentration of urine available for flow back out of the absorbent body to the liner. For example, the central wetness indicating zone 171 desirably comprises a mixture of hydrophilic fibers, such as a web of cellulosic fluff, and particles of a high-absorbency material commonly known as superabsorbent material. More particularly, the central wetness indicating zone 171 desirably comprises a mixture of cellulosic fluff, such as wood pulp fluff, and superabsorbent hydrogel-forming particles. Wood pulp fluff present in the central wetness indicating zone 171 is desirably a low density wood pulp fluff, e.g., having a density of less than or equal to about 0.15 g/cc (grams per cubic centimeter).

The concentration of superabsorbent material present in the central wetness indicating zone 171 is relatively low, particularly in comparison to the concentration of superabsorbent material present in each of the absorbent zones 173. For example, the concentration of superabsorbent material in the central wetness indicating zone 171 is desirably less than or equal to about fifteen percent by weight of the central wetness indicating zone. It is contemplated that the central wetness indicating zone 171 may even be devoid of superabsorbent material, thereby comprising only low density wood pulp fluff, without departing from the scope of this invention.

The absorbent zone 173 on each side of the central wetness indicating zone 171 also comprises a mixture of hydrophilic fibers, such as wood pulp fluff, and superabsorbent material. More particularly each absorbent zone 173 is desirably composed of a high density wood pulp fluff and superabsorbent material. For example, wood pulp fluff present in each absorbent zone 1 73 desirably has a density greater than about 0.15 g/cc, and more desirably greater than about 0.25 g/cc. The concentration of superabsorbent material present in the absorbent zones 173 desirably constitutes about forty percent by weight of the absorbent body 153.

One suitable type of wood pulp fluff from which the central wetness indicating zone 171 and the absorbent zones 173 may be constructed is identified with the trade designation CR1654, available from U.S. Alliance, Childersburg, Ala., U.S.A., and is a bleached, highly absorbent sulfate wood pulp containing primarily soft wood fibers. However, the wood pulp fluff can be exchanged with other hydrophilic fiber materials, such as synthetic, polymeric, or meltblown fibers or with a combination of meltblown fibers and natural fibers. Suitable superabsorbent material can be selected from natural, synthetic, and modified natural polymers and materials. The superabsorbent material can be inorganic material, such as silica gels, or organic compounds, such as cross-linked polymers. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany.

In accordance with one method of manufacturing such an absorbent body 153, wood pulp fluff and superabsorbent particles are generally concurrently deposited on a surface, such as a conveyor (not shown) to form the absorbent body. The concentration of wood pulp fluff deposited on the conveyor is approximately uniform throughout the central wetness indicating zone 171 and the absorbent zones 173, but the concentration of superabsorbent particles deposited on the conveyor is greater within the absorbent zones than in the central wetness indicating zone. The absorbent body 153 is then conveyed through a nip, defined by a roller press, for compaction. The roller press is contoured (e.g., hourglass-shaped) so that the size of the nip through which the central wetness indicating zone 171 passes is greater than the size of the nip through which the absorbent zones 173 pass. As a result of being passed through a narrower portion of the nip, each absorbent zone 173, and more particularly the wood pulp fluff in each absorbent zone, becomes compacted (i.e., densified) more than wood pulp fluff in the central wetness indicating zone 171 as the absorbent body is passed through the nip. Increasing the density of the wood pulp fluff in the absorbent zones 173 decreases the interstitial spacing between wood pulp fluff fibers, thereby reducing to the ability of the absorbent zones to quickly take in urine.

In an alternative method, a lesser concentration of wood pulp fluff may be deposited in the central wetness indicating zone 171 than in the absorbent zones 173. The absorbent body 153 can then be passed through a nip defined by a roller press having a uniform diameter. As a result, wood pulp fluff in the central wetness indicating zone 171 is compacted (e.g., densified) less than that in each absorbent zone 173.

In use, as the wearer of the training pants 121 urinates therein, urine penetrates the liner 151 and is received by the absorbent body 153, particularly in the central wetness indicating zone 171 of the absorbent body. Since the central wetness indicating zone 171 is composed of a lower density wood pulp fluff than the absorbent zones 173, urine is taken into the central wetness indicating zone faster than it is taken into the absorbent zones. However, since the central wetness indicating zone 171 has a substantially lower concentration of superabsorbent material therein, unabsorbed urine is maintained in the wood pulp fluff of the central wetness indicating zone, such as by being held within the interstitial spacing of the wood pulp fluff fibers, for a substantially longer duration than urine taken into the absorbent zones 173. Consequently, unabsorbed urine maintained in the central wetness indicating zone 171 is capable of flowing from the absorbent body 153 back through the liner 151 to provide a prolonged feeling of wetness against the wearer's skin, thereby indicating to the wearer that urination has occurred. Eventually, unabsorbed urine in the central wetness indicating zone 171 may be absorbed by superabsorbent material therein and/or it may flow laterally outward to the absorbent zones 173 and be absorbed therein.

### Experiment 2

An experiment was conducted to determine the amount of liquid available for flow back out of absorbent body samples after one minute, after five minutes, and after ten minutes following liquid delivery. The experiment entailed the use of samples having a length of 6.75 inches and a width of five inches. A first sample (i.e., Sample 1) was constructed to simulate the absorbent body 153 of this second embodiment. More particularly, the sample comprised a two inch wide central wetness indicating zone composed entirely of wood pulp fluff and absorbent zones each composed of a mixture of wood pulp fluff and superabsorbent material, with the concentration of superabsorbent material constituting about forty percent by weight of the sample. A second sample (i.e., Sample 2) was constructed to simulate an absorbent body of conventional training pants and was composed of a mixture of wood pulp fluff and superabsorbent material throughout, with the concentration of superabsorbent material constituting about forty percent by weight of the sample. The dry weight of each sample was measured and only samples that weighed within three percent of the targeted sample weight were tested.

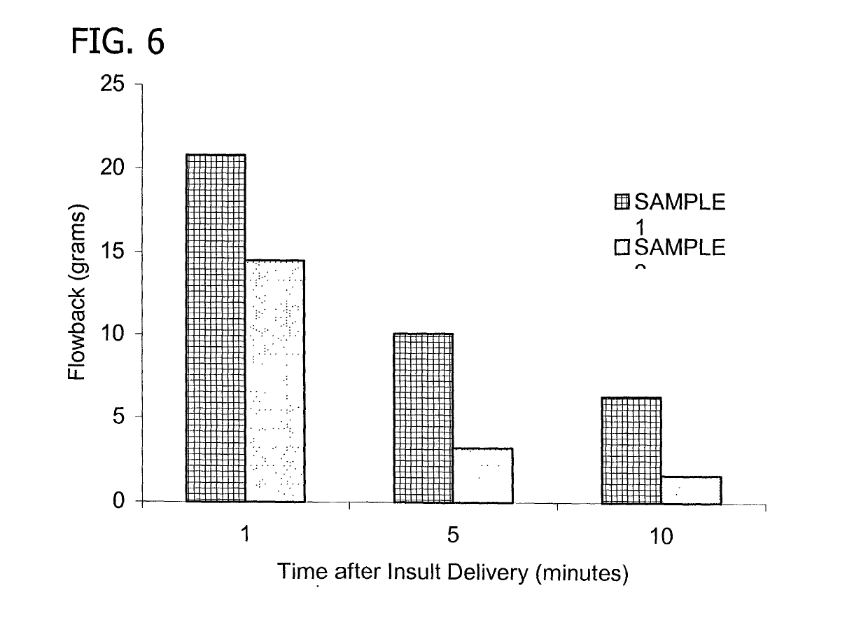

The samples were tested in accordance with the procedures set forth above in Experiment 1. The results of this experiment are illustrated in FIG. 6. Sample 1, which was constructed in accordance with the absorbent body of this second embodiment, maintained (referred to as flow back in FIG. 6) more than twenty grams of liquid available for flow back out of the sample for about one minute, compared to less than fifteen grams for the other sample (e.g., Sample 2), and maintained more than five grams of liquid available for flow back out of the sample for at least about ten minutes, compared to only about 1.5 grams for Sample 2. Thus, providing a central wetness indicating zone 171 results in maintaining an increased concentration of liquid capable of flowing back out of the absorbent body, thereby facilitating flow back through the liner 151 for a prolonged duration relative to an absorbent body of conventional training pants.

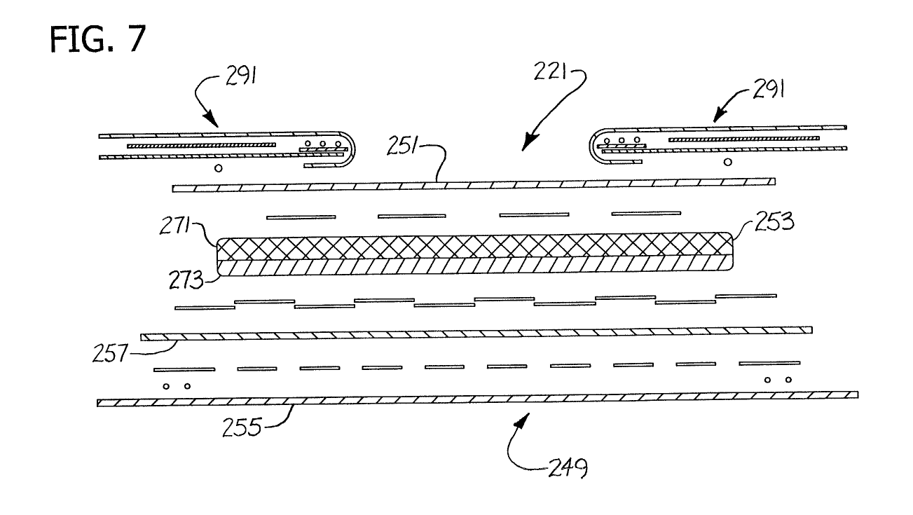

FIG. 7 illustrates training pants 221 of a third embodiment of the present invention similar to the training pants 21 of the first embodiment including a liner 251, an outer cover 249 having an outer layer 255 and an inner layer 257, an absorbent body 253 and containment flaps 191. The absorbent body 253 of this third embodiment is desirably composed of hydrophilic fibers, such as a web of cellulosic fluff, and a high-absorbency material commonly known as superabsorbent material. More particularly, the absorbent body 253 is desirably composed of cellulosic fluff, such as wood pulp fluff, and superabsorbent hydrogel-forming particles. One suitable type of wood pulp fluff is identified with the trade designation CR1654, available from U.S. Alliance, Childersburg, Ala., U.S.A., and is a bleached, highly absorbent sulfate wood pulp containing primarily soft wood fibers. However, the wood pulp fluff can be exchanged with other hydrophilic fiber materials, such as synthetic, polymeric, or meltblown fibers or with a combination of meltblown fibers and natural fibers.

Suitable superabsorbent materials can be selected from natural, synthetic, and modified natural polymers and materials. The superabsorbent materials can be inorganic materials, such as silica gels, or organic compounds, such as cross-linked polymers. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany.

As shown in FIG. 7, the absorbent body 253 has an inner zone 271 (broadly, a first zone) and an outer zone 273 (broadly, a second zone). The inner zone 271 is nearer the liner 251 than the outer zone 273 and desirably has a lower absorbent capacity per unit weight than the outer zone. More particularly, the inner zone 271 is desirably devoid of superabsorbent material, although some superabsorbent material may be present in the inner zone as long as the concentration of superabsorbent material therein is substantially lower than the concentration of superabsorbent material present in the outer zone 273.

As an example, the outer zone 273 may constitute about twenty percent to about seventy percent by weight of the absorbent body 253 and about one percent to about seventy percent of the absorbent body thickness. The concentration of superabsorbent material in the outer zone 273 desirably constitutes about forty percent to about one-hundred percent by weight of the outer zone and about five percent to about fifty percent by weight of the entire absorbent body 253, and more desirably about forty percent by weight of the entire absorbent body. While the inner and outer zones 271, 273 of the embodiment shown in FIG. 7 have substantially the same width, it is contemplated that the widths, and the lengths, of the inner and outer zones may differ without departing from the scope of this invention.

In one method of manufacturing the absorbent body 253 of this third embodiment, the inner and outer zones 271, 273 are integrally formed by depositing wood pulp fluff and superabsorbent particles on a surface, such as a conveyor (not shown) for accumulation thereby to form the absorbent body. The concentration of superabsorbent material deposited on the conveyor during an initial time period corresponding to formation of the outer zone 273 is substantially greater than the concentration of superabsorbent material deposited on the conveyor for a subsequent time period corresponding to formation of the inner zone 271. Where the outer zone 273 is composed entirely of superabsorbent material, no wood pulp fluff is deposited during the initial time period. Rather, deposition of the wood pulp fluff is initiated during the subsequent time period. Likewise, where the inner zone 271 is to be devoid of superabsorbent material, deposition of superabsorbent material ceases after the initial time period. It is understood that the absorbent body 253 may alternatively be formed in reverse order, such as by forming the outer zone 273 first and then the inner zone 271, without departing from the scope of this invention. The absorbent body 253 may be conveyed through a nip, defined by a roller press, for compaction. In an alternative method of manufacturing the absorbent body 253, the inner and outer zones 271, 273 may be formed as separate layers and then overlaid to form the absorbent body.

As the wearer of the training pants 221 urinates therein, urine penetrates the liner 251 and is received by the absorbent body 153, and more particularly by the inner zone 271 of the absorbent body. Since the inner zone 271 has a lower concentration of superabsorbent material therein than the outer zone 273, unabsorbed urine is maintained therein, such as by being entrained in the interstitial spacing of the wood pulp fluff fibers, for a prolonged duration. The maintenance of unabsorbed urine in the inner zone 271 in this manner facilitates the flow of urine from the inner zone back through the liner 251 to provide a prolonged feeling of wetness against the wearer's skin, thereby indicating to the wearer that urination has occurred. Eventually, urine in the inner zone 271 flows to the outer zone 273 for absorption by superabsorbent material therein.

### Experiment 3

An experiment was conducted to determine the amount of liquid available for flow back out of various absorbent body samples after one minute, after five minutes, and after of ten minutes following liquid delivery therein. The experiment entailed the use of samples having a length of 6.75 inches and a width of five inches. A first sample (i.e., Sample 1) was constructed of a single layer in accordance with the absorbent body 253 of this third embodiment, having an inner zone composed entirely of wood pulp fluff and an outer zone composed of a homogeneous mixture of wood pulp fluff and superabsorbent material. The outer zone constituted about twenty-five percent of the sample thickness and about forty percent by weight of the sample. A second sample (i.e., Sample 2) constructed in accordance with the absorbent body 253 was constructed of two layers, with one layer defining the inner zone and being composed entirely of wood pulp fluff and the other layer defining the outer zone and being composed of a homogeneous mixture of wood pulp fluff and superabsorbent material. The superabsorbent material constituted about forty percent by weight of the sample and about sixty-one percent by weight of the outer zone. The outer zone of the second sample constituted about sixty-five percent of the sample thickness. A third sample (i.e., Sample 3) was constructed to simulate an absorbent body of conventional training pants and was composed of a homogeneous mixture of wood pulp fluff and superabsorbent material, with the superabsorbent material comprising about forty percent by weight of the sample. The dry weight of each sample was measured and only samples that weighed within three percent of the targeted sample weight were tested.

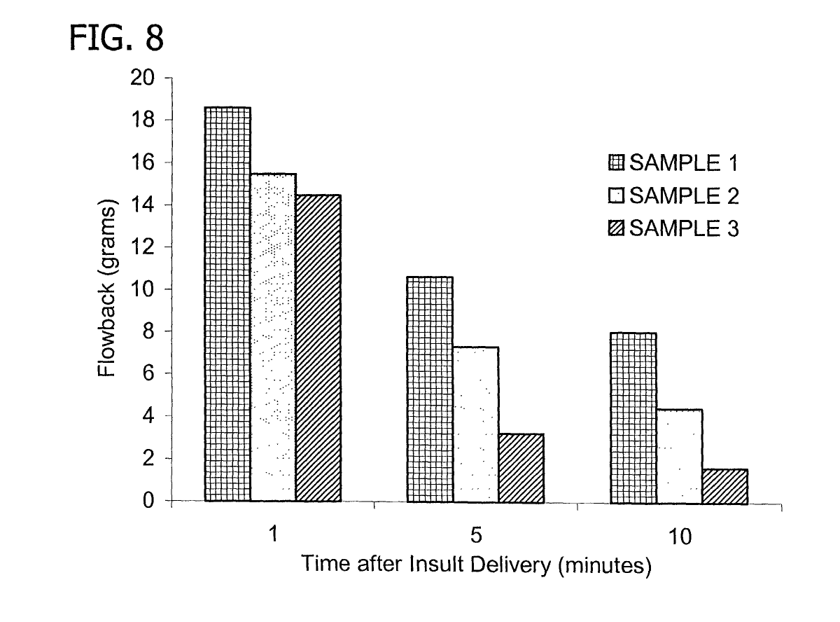

Each sample was tested in the manner described above with respect to Experiment 1. The results of the experiment are illustrated in FIG. 8. Sample 1, in which the outer zone containing superabsorbent material constituted only about twenty-five percent of the sample thickness, maintained (referred to as flow back in FIG. 8) a greater amount of liquid therein available for flow back, and for a longer duration, than the other samples. Sample 2, in which the outer zone containing superabsorbent material comprised about sixty-five percent of the sample thickness, also maintained a greater amount of liquid for flow back, and for a longer duration, than the third sample (i.e., Sample 3) constructed to simulate an absorbent body of conventional training pants.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A disposable absorbent article for personal wear, said disposable absorbent article comprising:
a generally liquid permeable liner adapted for contiguity with the wearer's skin;
an outer cover; and
an absorbent body between the liner and the outer cover for absorbing liquid body waste penetrating the liner, said absorbent body comprising a first zone and a second zone, the first zone having a lower absorbent capacity per unit weight than the second zone and facilitating the flow of liquid body waste from the first zone back through the liner for indicating to the wearer the release of liquid body waste into said article.

2. A disposable absorbent article as set forth in claim 1 wherein the absorbent body comprises a wetness indicating zone defining said first zone and an absorbent zone defining said second zone, the absorbent zone being adjacent the wetness indicating zone.

3. A disposable absorbent article as set forth in claim 1 wherein the absorbent body comprises a wetness indicating zone defining said first zone and a pair of absorbent zones defining said second zone and being adjacent the wetness indicating zone, said wetness indicating zone being intermediate said absorbent zones.

4. A disposable absorbent article as set forth in claim 3 wherein the wetness indicating zone comprises hydrophilic fibers and each absorbent zone comprises a mixture of hydrophilic fibers and superabsorbent material.

5. A disposable absorbent article as set forth in claim 4 wherein the wetness indicating zone comprises a mixture of hydrophilic fibers and superabsorbent material, the concentration of superabsorbent material in the wetness indicating zone being less than the concentration of superabsorbent material in each absorbent zone.

6. A disposable absorbent article as set forth in claim 4 wherein the wetness indicating zone comprises a low density cellulosic fluff and each absorbent zone comprises a mixture of a high density cellulosic fluff and superabsorbent material.

7. A disposable absorbent article as set forth in claim 5 wherein the wetness indicating zone has a concentration of superabsorbent material of less than or equal to about fifteen percent by weight thereof.

8. A disposable absorbent article as set forth in claim 7 wherein each absorbent zone has a concentration of superabsorbent material of at least about forty percent by weight of the absorbent body.

9. A disposable absorbent article as set forth in claim 3 wherein the wetness indicating zone has a width of about thirty percent to about seventy percent of the total width of the absorbent body.

10. A disposable absorbent article as set forth in claim 1 wherein the absorbent body comprises an inner zone defining said first zone and an outer zone defining said second zone, the inner zone being nearer the liner than the outer zone.

11. A disposable absorbent article as set forth in claim 10 wherein the inner zone comprises hydrophilic fibers and the outer zone comprises superabsorbent material.

12. A disposable absorbent article as set forth in claim 11 wherein the inner zone comprises a mixture of hydrophilic fibers and superabsorbent material, the outer zone also comprising a mixture of hydrophilic fibers and superabsorbent material, the concentration of superabsorbent material in said inner zone being less than the concentration of superabsorbent material in said outer zone.

13. A disposable absorbent article as set forth in claim 11 wherein the inner zone comprises only hydrophilic fibers.

14. A disposable absorbent article as set forth in claim 11 wherein the outer zone comprises only superabsorbent material.

15. A disposable absorbent article as set forth in claim 9 wherein the outer zone has a thickness of between about one percent and about seventy percent of the thickness of the absorbent body.

16. A disposable absorbent article as set forth in claim 10 wherein the outer zone has a weight of between about twenty percent and about seventy percent by weight of the absorbent body.

17. A disposable absorbent article as set forth in claim 16 wherein the outer zone has a weight of between about thirty-five percent and about fifty percent by weight of the absorbent body.

18. A disposable absorbent article as set forth in claim 10 wherein the outer zone has a concentration of superabsorbent material of at least about forty percent by weight thereof.

19. Toilet training pants comprising:
an anterior region, a posterior region and a crotch region disposed longitudinally therebetween, said anterior region, posterior region and crotch region being integrally formed and configured to define a central waist opening and a pair of leg openings of said pants, the crotch region extending generally laterally between said leg openings;
a generally liquid permeable liner extending from the anterior region through the crotch region to the posterior region of said pants and being if adapted for contiguity with the wearer's skin;
an outer cover; and
an absorbent body between the liner and the outer cover for absorbing liquid body waste penetrating the liner, said absorbent body comprising a first zone and a second zone, the first zone having a lower absorbent capacity per unit weight than the second zone and being capable of facilitating the flow of liquid body waste from the first zone back through the liner for indicating to the wearer the release of liquid body waste into said training pants.

20. Training pants as set forth in claim 19 wherein the absorbent body comprises a wetness indicating zone defining said first zone and an absorbent zone defining said second zone, the absorbent zone being adjacent the wetness indicating zone in side-by-side, abutting relation therewith.

21. Training pants as set forth in claim 19 wherein the absorbent body comprises a wetness indicating zone defining said first zone and a pair of absorbent zones defining said second zone, the central wetness indicating zone being disposed between the absorbent zones in side-by-side, abutting relation therewith.

22. Training pants as set forth in claim 21 wherein the wetness indicating zone comprises hydrophilic fibers and each absorbent zone comprises a mixture of hydrophilic fibers and superabsorbent material.

23. Training pants as set forth in claim 22 wherein the wetness indicating zone comprises a mixture of hydrophilic fibers and superabsorbent material, the concentration of superabsorbent material in the wetness indicating zone being less than the concentration of superabsorbent material in each absorbent zone.

24. Training pants as set forth in claim 22 wherein the wetness indicating zone comprises a low density cellulosic fluff and each absorbent zone comprises a mixture of a high density cellulosic fluff and superabsorbent material.

25. Training pants as set forth in claim 23 wherein the wetness indicating zone has a concentration of superabsorbent material of less than or equal to about fifteen percent by weight thereof.

26. Training pants as set forth in claim 25 wherein each absorbent zone has a concentration of superabsorbent material of at least about forty percent by weight thereof.

27. Training pants as set forth in claim 21 wherein the central wetness indicating zone has a width of about thirty percent to about seventy percent of the width of the absorbent body.

28. Training pants as set forth in claim 19 wherein the absorbent body comprises an inner zone defining said first zone and an outer zone defining said second zone, the inner zone being nearer the liner than the outer zone.

29. Training pants as set forth in claim 28 wherein the inner zone comprises hydrophilic fibers and the outer zone comprises superabsorbent material.

30. Training pants as set forth in claim 29 wherein the inner zone comprises a mixture of hydrophilic fibers and superabsorbent material, the outer zone also comprising a mixture of hydrophilic fibers and superabsorbent material, the concentration of superabsorbent material in said inner zone being less than the concentration of superabsorbent material in said outer zone.

31. Training pants as set forth in claim 29 wherein the inner zone comprises only hydrophilic fibers.

32. Training pants as set forth in claim 29 wherein the outer zone comprises only superabsorbent material.

33. Training pants as set forth in claim 28 wherein the outer zone has a thickness of between about on e percent and about seventy percent of the thickness of the absorbent body.

34. Training pants as set forth in claim 28 wherein the outer zone has a weight of between about twenty percent and about seventy percent of the weight of the absorbent body.

35. Training pants as set forth in claim 34 wherein the outer zone has a weight of between about thirty-five percent and about fifty percent of the weight of the absorbent body.

36. A disposable absorbent article as set forth in claim 29 wherein the outer zone has a concentration of superabsorbent material of at least about forty percent by weight of the absorbent body.

37. A method of manufacturing an absorbent body for a disposable absorbent article of the type having a liner, an outer cover and an absorbent body between the liner and outer cover for absorbing liquid body waste penetrating the liner, the absorbent body having a width and comprising a wetness indicating zone disposed between absorbent zones in generally side-by-side abutting relation therewith, said method comprising:
concurrently depositing hydrophilic fibers and superabsorbent material on a surface to form the absorbent body, said hydrophilic fibers being deposited generally uniformly across the absorbent body, the concentration of superabsorbent material deposited in the wetness indicating zone being lower than the concentration of superabsorbent material deposited in the absorbent zones.

38. A method as set forth in claim 37 wherein the superabsorbent material is deposited solely in the absorbent zones.

39. A method as set forth in claim 37 further comprising the step of compacting the absorbent body, said compacting step comprising compacting the hydrophilic fibers in the wetness indicating zone less than the hydrophilic fibers in the absorbent zones.

40. A method of manufacturing an absorbent body for a disposable absorbent article of the type having a liner, an outer cover and an absorbent body between the liner and outer cover for absorbing liquid body waste penetrating the liner, the absorbent body having a width and comprising a wetness indicating zone disposed between absorbent zones in generally side-by-side abutting relation therewith, said method comprising:
concurrently depositing hydrophilic fibers and superabsorbent material on a surface to form the absorbent body, the concentration of hydrophilic fibers deposited in the wetness indicating zone being greater than the concentration of fibers deposited in the absorbent zones, the concentration of superabsorbent material deposited in the wetness indicating zone being lower than the concentration of superabsorbent material deposited in each absorbent zone.

41. A method as set forth in claim 40 wherein the superabsorbent material is deposited solely in the absorbent zones.

42. A method as set forth in claim 40 further comprising the step of uniformly compacting the absorbent body whereby the hydrophilic fibers in the absorbent zones are more compacted than the hydrophilic fibers in the wetness indicating zone as the absorbent body is uniformly compacted.

43. A disposable absorbent article for personal wear, said disposable absorbent article comprising:
a generally liquid permeable liner adapted for contiguity with the wearer's skin;
an outer cover; and
an absorbent body between the liner and outer cover for absorbing liquid body waste penetrating the liner, said absorbent body comprising a mixture of hydrophilic fibers and superabsorbent material, the mixture having a concentration of superabsorbent material therein of between about one percent and about seven percent by weight of the mixture to facilitate the flow of liquid body waste from the absorbent body back through the liner for indicating to the wearer the release of liquid body waste into said article.

44. A disposable absorbent article as set forth in claim 43 wherein the absorbent body is capable of maintaining at least about 1 gram of unabsorbed liquid body waste therein per 1 gram of absorbent body weight for a period of up to about ten minutes following the release of liquid body waste into said article.

45. A disposable absorbent article as set forth in claim 44 wherein the absorbent body is capable of maintaining at least about 1.5 grams of unabsorbed liquid body waste therein per 1 gram of absorbent body weight for a period of up to about ten minutes following the release of liquid body waste into the article,

46. A disposable absorbent article for personal wear, said disposable absorbent article comprising:
a generally liquid permeable liner adapted for contiguity with the wearer's skin;
an outer cover; and
an absorbent body between the liner and outer cover for absorbing liquid body waste penetrating the liner, said absorbent body having a width of at least about sixty percent of the width of said article and comprising a mixture of hydrophilic fibers and superabsorbent material, the mixture having a concentration of superabsorbent material therein of between about one percent and about fifteen percent by weight of the mixture to facilitate the flow of liquid body waste from the absorbent body back through the liner for indicating to the wearer the release of liquid body waste into said article.

47. A disposable absorbent article as set forth in claim 46 wherein the mixture has a concentration of superabsorbent material of between about one percent and about seven percent by weight of the mixture.

48. A disposable absorbent article as set forth in claim 46 wherein the absorbent body is capable of maintaining at least about 1 gram of unabsorbed liquid body waste therein per 1 gram of absorbent body weight for a period of up to about ten minutes following the release of liquid body waste into the article.

49. A disposable absorbent article as set forth in claim 48 wherein the absorbent body is capable of maintaining at least about 1.5 grams of unabsorbed liquid body waste therein per I gram of absorbent body weight for a period of up to about ten minutes following the release of liquid body waste into the article.
